(19) United States
(12) Patent Application Publication
(10) Pub. No.: US 2008/0233201 A1
(43) Pub. Date: Sep. 25, 2008

(54) Method for Preparing Calibrated Biodegradable Microspheres

(76) Inventors: Audrey Royere, Angers (FR); Didier Bazile, Angers (FR); Jerome Bibette, Paris (FR)

Correspondence Address: YOUNG & THOMPSON, 209 Madison Street, Suite 500, ALEXANDRIA, VA, 22314, US

(21) Appl. No.: 10/591,131

(22) PCT Filed: Mar. 3, 2005
(86) PCT No.: PCT/FR2005/000511

§ 371 Date: Sep. 26, 2006

(30) Foreign Application Priority Data
Mar. 3, 2004 (FR) 0402204

(51) Int. Cl. A61K 9/14; A61K 38/00; A61K 31/70

(52) U.S. Cl. 424/501; 514/2; 514/44

(57) ABSTRACT

The invention relates to a method for preparing monodispersed biodegradable microspheres consisting in a) preparing an emulsion containing at least one polymeric phase and at least one aqueous phase at a viscosity ratio between the dispersed polymeric phase and the aqueous phase ranging from 0.12 to 10, b) exposing the thus obtained emulsion to a controlled laminar shear strength, c) removing a solvent from the polymeric phase and in d) isolating the thus obtained microspheres. The use of the prepared microspheres is also disclosed.

The invention relates to the pharmaceutical industry. More precisely, it relates to the preparation of monodisperse biodegradable microspheres, especially for the administration of pharmaceutically active ingredients.

It is known to encapsulate pharmaceutically active ingredients in microspheres in order to facilitate their administration or prevent their degradation in vivo.

Microencapsulation consists in coating solid or liquid substances in such a manner as to make them into particles whose size varies from 0.1 to 1000 μm.

In this context, the use of biodegradable microspheres that deliver the active ingredient over a prolonged period was especially envisaged.

Various techniques for preparing biodegradable microspheres are known.

Thus, U.S. Pat. No. 5,643,607 discloses microcapsules for the prolonged administration of hydrophilic active ingredients, in particular peptides. The microcapsules are prepared by microencapsulation of an emulsion whose dispersed aqueous phase contains the active ingredient and whose continuous phase contains a polymer.

However, it is observed that the release kinetics of the active ingredient contained in these microspheres is non-homogeneous. This effect is due to the fact that the microspheres have a broad particle size distribution. The release of the active ingredient from the microspheres is based on diffusion effects and is therefore generally slowed down for microspheres of increasing size, being extended over longer periods.

A method for preparing monodisperse microspheres consists in passing a polymer solution through a nozzle subjected to vibration, each of the vibrations bringing about the breakage of the flow leaving the nozzle to form a droplet (Berkland et al. J. Controlled Release 73 (2001), 59-74). This method is complex and long and has a low yield. In addition, it seems difficult to transfer to an industrial scale. Furthermore, it does not always permit homogeneous distribution of the active ingredient inside the microcapsules, since it is based on a phenomenon of instantaneous precipitation.

Therefore, an object of the present invention is to provide a method for preparing monodisperse biodegradable microcapsules of controlled size, which are intended especially for transporting both water-soluble and lipid-soluble active ingredients.

From patent FR 2 747 321 is known a method for preparing monodisperse emulsions by controlled laminar shearing in a device of the Couette type. However, this method aims only at providing lipid emulsions and not complex systems in which the organic phase comprises a polymer and an organic solvent.

The invention is based principally on the finding that an emulsion containing at least one polymeric organic phase can be obtained when the ratio of the viscosities between the dispersed phase and the continuous phase (η_{org}/η_{aq} in the case of a direct emulsion or η_{aq}/β_{org} in the case of an inverse emulsion) is from 0.1 to 10.

Thus, the invention is directed more precisely to a method for preparing monodisperse biodegradable microspheres comprising the steps of:
a) preparing an emulsion comprising at least one polymer phase and at least one aqueous phase; the ratio of the viscosities between the dispersed phase and the continuous phase must be from 0.1 to 10;
b) subjecting the emulsion obtained to controlled laminar shearing;
c)removing the solvent from the polymer phase; and
d) isolating the so obtained microspheres.

In the present invention, the term “microspheres” denotes spherical units having a diameter of from 0.1 μm to 1000 μm, more especially from 0.7 μm to 30 μm.

Microspheres according to the invention are constituted by a polymer-based matrix. They thus lend themselves particularly well to the administration of heat-sensitive active ingredients, for example proteins or polypeptides. While a lipid phase is converted into liquid by heating, the formation of the polymer microspheres is based on the dissolution of the polymer in an organic solvent. When the solvent has been removed, the polymer components of the microspheres form a uniform matrix in which an active ingredient can be encapsulated. The polymer microspheres can therefore be manufactured without increasing the temperature.

Depending on the solubility of the active ingredient, the latter may be encapsulated directly in the polymer phase, that is to say, inside microdroplets of aqueous phase which are contained in the polymer matrix of the microspheres. Generally, it will be encapsulated in the polymer matrix when the active ingredient is lipid-soluble. In contrast, it is encapsulated in the internal aqueous phase when the active ingredient is water-soluble. Some active ingredients have a low solubility both in water and in non-polar solvents. In that case, the active ingredient can be dispersed in the solid state in the polymer solution.

The administration of active ingredients that are neither lipid-soluble nor water-soluble is particularly tricky when using the known galenical forms. The microspheres according to the invention therefore appear to be of particular value for the administration of those active ingredients.

In the present Application, “biodegradable” means a material which is degraded in a biological medium and whose degradation products are removed by renal filtration or metabolized. Biodegradable polymers are defined as being synthetic or natural polymers that are degradable in vivo in an enzymatic or non-enzymatic manner to produce non-toxic degradation products.

This degradation generally takes place over a period ranging from a few weeks to a few months (example: PGA-TMC is absorbed in 7 months while L-PLA has a degradation period of approximately 2 years).

The degradation time of a polymer depends on its type, and therefore on the chemical nature of the monomer units, but also on its degree of polymerization and its crystallinity. In addition, apart from the material, it will depend particularly on the surface area of material accessible to enzymes or other degrading substances. Thus, the more finely divided the material is, the more rapidly it will be degraded.

The microspheres are degraded in such a manner that the amount of polymer accumulated in the organism does not exceed an amount equivalent to 20 times the dose of polymer administered per administration. Preferably, the amount of polymer accumulated in the organism does not exceed an amount equivalent to 10 times the dose of polymer administered per administration.

The interval separating two successive administrations of microspheres according to the invention is generally at least one day, preferably from 1 day to 30 days, and in particular from 5 to 14 days.

Thus, the microspheres are prevented from accumulating in the body.

The microspheres according to the present invention comprise a polymer matrix in which one or more active ingredients or droplets of aqueous solution, which may themselves contain one or more active ingredients, may be distributed.

The active ingredient(s) may be, independently of each other, water-soluble or poorly water-soluble, lipid-soluble or poorly lipid-soluble or also both poorly lipid-soluble and poorly water-soluble.

In the case of compositions whose dispersed phase comprises an internal aqueous phase, it is possible, for example, to carry hydrophilic active ingredients alone or in combination with the poorly water-soluble active ingredients.

The active ingredient may be especially a pharmaceutical, veterinary, plant-protective, cosmetic or agroalimentary active ingredient. Furthermore, it may be a detergent, a nutrient, an antigen or a vaccine. Preferably, it is a pharmaceutically active ingredient.

Preferably, the pharmaceutically active ingredient is selected from the groups constituted by antibiotics, hypolipidaemics, antihypertensives, antiviral agents, beta blockers, bronchodilators, cytostatics, psychotropic agents, hormones, vasodilators, anti-allergics, analgesics, antipyretics, antispasmodics, anti-inflammatories, anti-angiogenics, antibacterials, anti-ulcerants, antifungals, antiparasitics, antidiabetics, anti-epileptics, anti-Parkinsons, antimigraines, anti-Alzheimers, anti-acneics, antiglaucomic agents, anti-asthmatics, neuroleptics, antidepressants, anxiolytics, hypnotics, normothymics, sedatives, psychostimulants, anti-osteoporosis agents, anti-arthritics, anticoagulants, antipsoriasis agents, hyperglycaemics, orexigenics, anorexigenics, anti-asthenics, anticonstipation agents, antidiarrhoeals, anti-trauma agents, diuretics, myorelaxants, enuresis medicaments, erection disorder medicaments, vitamins, peptides, proteins, anticancer agents, nucleic acids, RNA, oligonucleotides, ribozymes and DNA.

In addition, it may prove advantageous to combine the active ingredient(s) with an agent modulating absorption by the oral route or with an enzyme inhibitor, for example a P-glycoprotein inhibitor or a protease inhibitor.

The term “monodisperse” is intended to denote a population of microspheres, the diameter of each microsphere of which is very close to the average diameter of the population. A population is called “monodisperse” when the polydispersity is less than or equal to 40%, and preferably of the order of from 5 to 30%, for example from 15 to 25%. The polydispersity is then defined as being the ratio of the standard deviation to the median of the distribution of the diameter, represented by volume, of the droplets or globules.

The monodisperse microspheres according to the present invention are obtained by subjecting to controlled shearing an emulsion comprising, as the dispersed phase, droplets of polymer phase (which may or may not contain droplets of internal water) comprising one or more active ingredients. Moreover, parametrable and controllable shearing permits control of the size of the microspheres and thereby of the release of the active ingredient and its removal from the organism.

Preferably, this step is implemented in an apparatus of the Couette type. Microspheres are thus obtained whose size distribution is narrow and homogeneous.

The method according to the invention for preparing the microspheres has the advantage of being a simple method and of using only a small amount of solvent. It can be readily transferred to an industrial scale.

In addition, this method has a high yield of encapsulation of active ingredient in the microspheres. What is meant by encapsulation yield is the ratio between the encapsulated active ingredient and the active ingredient used. This may be optimized in the method by a partition coefficient between the aqueous phase and the organic phase favourable to the dissolution of the active ingredient in the organic phase and a high concentration of organic phase in the emulsion. To be more precise, the method consists in preparing, in a first step, an emulsion comprising at least one organic phase and at least one aqueous phase.

If there is an organic phase and an aqueous phase, a direct single emulsion is prepared.

The term “direct emulsion” denotes an emulsion in which an organic phase is dispersed in an aqueous phase. In contrast, in an “inverse” emulsion, an aqueous phase is dispersed in an organic phase.

The direct emulsion is especially useful in encapsulating a lipid-soluble active ingredient (dissolved in the organic phase).

The production of microspheres from double emulsions is, however, also possible. These emulsions comprise two aqueous phases: a so-called “internal” aqueous phase, which is dispersed in the organic phase, which is itself dispersed in the so-called “external” aqueous phase. The internal aqueous phase therefore permits the dissolution of hydrophilic active ingredients and in particular of fragile active ingredients, such as proteins or polypeptides, for example.

Thus, depending on whether it is desired to encapsulate lipophilic or hydrophilic active ingredients, either a direct single emulsion or a double emulsion W/Org/W will be used. The double emulsion is also a means of obtaining microspheres encapsulating several active ingredients, for example, a combination of a hydrophilic active ingredient (dissolved in the internal aqueous phase) and a hydrophobic active ingredient (dissolved in the organic solution containing the polymer).

The organic phase of the emulsion contains at least one biodegradable polymer dissolved in an organic solvent.

The organic phase of the emulsions advantageously contains from 5 to 30% of at least one biodegradable polymer and preferably from 10 to 20% by mass of the total mass of the organic phase.

The polymer is selected from biodegradable polymers that are non-toxic to humans and animals. It is also advantageously inert with respect to the active ingredient and insoluble in water.

The biodegradable polymer(s) used are preferably polymers approved for use in the administration route considered (for example, parenteral). Preferably, polymers whose degradation products can be readily removed by the organism will be used as biodegradable polymers.

Among those polymers, one may especially mention those derived from lactic acid, and in particular from the family of the α-hydroxy acids, such as PLGA (polylactic glycolic acid). These polymers are approved for parenteral use in humans. They also have kinetics of degradation in the organism suitable in terms of the release of the active ingredient. The degree of crystallinity of the polymer will have a direct influence on its hydrophilic character and also on the rapidity of its degradation in vivo.

These polymers are degraded in the organism by a non-specific chemical hydrolysis mechanism or by enzyme degradation. The monomers resulting therefrom are metabolized and lead to degradation products which are mainly removed via the respiratory route in the form of carbon dioxide and water.

Thus, it is possible to use for the implementation of the present invention polymers selected from poly(α-hydroxy acids), the aliphatic polyesters of poly(α-hydroxy acids), of poly(ε-caprolactones)—PCL, of polydioxanones—PDO, polyorthoesters, polyanhydrides, polycyanoacrylates, polyurethanes, polypeptides or poly(amino acids), modified polysaccharides, cellulose, polycarbonates, polydimethylsiloxanes and poly(vinyl acetates) and their derivatives and copolymers.

The polymers of the class of the poly(α-hydroxy acids) are polyesters whose repeating units are derived from α-hydroxy acids, such as poly(glycolides) (PGA), poly(lactides) (PLA), poly(lactide-co-glycolides) (PLAGA or PLGA), glycolide-co-trimethylene carbonate copolymers, or polyglyconates, (PGA-TMC). They are commercially available (for example, under the names Resomer® and Medisorb®).

Other polymers may be envisaged, such as the terpolymers resulting from the polymerization of glycolide with trimethylene carbonate and p-dioxanone, or block copolymers, such as polyethylene glycol-poly(α-hydroxy acids) (PLA-PEG, PLGA-PEG) or methoxy polyethylene glycol-poly(α-hydroxy acids).

In the same way as for α-hydroxy acids, the degree of crystallinity of the polymer will have a direct effect on its hydrophilic character and also on the rapidity of its degradation in vivo.

ε-caprolactone is an ester of hydroxy-6-caproic acid. Poly(ε-caprolactone) and its copolymers obtained with lactic acid are semi-crystalline polymers used in the composition of controlled-release medicament forms. These polymers are degraded in the organism in a manner similar to that of the PLAs and PLGAs (non-enzymatic degradation). Such a polymer is marketed under the name Lactel®.

The polydioxanones—PDO are polyether esters obtained by opening the ring of p-dioxanone.

Some active ingredients are unstable, especially those which are subject to rapid hydrolysis. It is therefore contraindicated to use polymers that retain water. In that case, polymers that are more hydrophobic and that are degraded by surface erosion, such as polyorthoesters and polyanhydrides, are to be preferred.

Polyorthoesters are compounds resulting from the condensation of 2,2-diethoxytetrahydrofuran with a diol. These polymers have, as degradation products, acid compounds that catalyse the degradation process. Degradation therefore accelerates in the course of time. They are marketed under the name Chronomer® and Alzamer®, for example.

Polyanhydrides are compounds derived from sebacic acid p(SA) and bis-p(carboxyphenoxy)propane p(CPP). The sebacic acid may also be combined with a fatty acid dimer (oleic acid: p(FAD-SA). Their degradation time may vary from a few days to a few years depending on the degree of hydrophobicity of the monomer used. They are degraded owing to surface erosion and have excellent biocompatibility.

Preferred polycyanoacrylates are polycyanoacrylates having a long alkyl chain, which are degraded slowly and cause little inflammatory reaction of the tissues. Such polymers are available under the name Desmolac® (BAYER).

Polypeptides or poly(amino acids) are polyamides resulting from the condensation of molecules naturally present in the organism. In order to obtain a material which is hydrolysed gradually in the course of time, the polymers resulting from simple amino acids (hydrophilic) and from hydrophobic derivatives of amino acids, such as the methyl or benzyl esters of aspartic acid, are preferred.

The assumed mechanism of degradation is first of all hydrolysis of the ester functions (disulphide bridges) giving water-soluble macromolecules, then a process of diffusion towards the liver and kidneys in which the peptide bonds are broken by enzyme attack. Ultramid A4 Naturel (BASF) may be mentioned by way of example of this class of polymer.

Of the cellulose derivatives, mention may be made more especially of methylcellulose and ethylcellulose, which are marketed, for example, under the name Blanose®, Ethocel® (Dow Cemica), Pharmacoat® 603 or 606 (ShinEtsu Chemical), and Aqualon EC® (Aqualon company).

Poly(trimethylene carbonate) (Poly(TMC)) and poly(propylene carbonate), available under the name Araconate 5 000, may be mentioned as polycarbonates.

Of the poly(vinyl acetates), the copolymer of ethylene and vinyl acetate (EVA), available, for example, under the name Coathylene® (plast-Labor SA) is particularly preferred.

The polymers present in the organic phase preferably have an average molecular mass of from 50 to 500 kDaltons, and in particular from 100 to 200 kDaltons.

In an entirely preferred manner, the microspheres are prepared from the family of the PLGAs. Of the family of these polymers, PLGA 75/25 (lactic/glycolic) or 85/15, which are sold under the name “High IV”, having a molecular weight of from 110 to 160 kDaltons, have been found to be particularly suitable.

These polymers have different hydrophobicity properties depending on the proportion of lactic acid units. Thus, the more the concentration of lactic acid increases, the more hydrophobic the PLGA will be.

On the other hand, the higher the proportion of lactic acid, the longer will be the degradation kinetics of the polymer. This characteristic of the polymer affects the release kinetics of the encapsulated active ingredient. These characteristics, which differ from one PLGA to another, therefore make it possible to use the one or the other, or even a mixture, of these copolymers, depending on the desired release characteristics.

Furthermore, the PLGA copolymers are soluble in several organic solvents, such as chloroform, dichloromethane or ethyl acetate, while they are practically insoluble in water.

Finally, this type of polymer is degraded by hydrolysis and the products of the reaction are metabolized to form CO₂ and H₂O, which are removed during breathing.

The organic solvent used for the preparation of the microspheres is preferably approved for parenteral use in humans. It is also selected to permit good dissolution of these polymers, preferably at ambient temperature.

In addition, the organic solvent preferably exhibits some solubility in water, for one method of performing the later removal of the solvent consists in extracting the solvent by diffusion in a large volume of water. It is also possible to remove the solvent by evaporation. Such solvents include, for example, ethyl acetate and dichloromethane.

Ethyl acetate is a volatile colourless solvent which is moderately soluble in water (8.7 g/100 g of water at 20° C.) and whose water-solubility decreases when the temperature increases. It is also tolerated well by the organism and does not pose any particular problems in terms of the environment.

Advantageously, the organic phase of the emulsion is saturated with water and, conversely, the aqueous phase(s) is(are) saturated with organic solvent in order to limit the escape of water from the aqueous phase towards the organic phase, and vice versa.

The organic phase may also advantageously contain an active ingredient which is lipophilic or poorly lipid-soluble and poorly water-soluble.

The aqueous phase of the-direct emulsion as well as the so-called “external” aqueous phase of the double emulsion preferably contain other agents apart from water. Preferably, these agents are approved for parenteral use. Thus, stabilizing agents, generally surfactants, are preferably added in order to increase the stability of the emulsion.

Non-ionic surfactants such as PVA (polyvinyl alcohol), or non-ionic surfactants such as polysorbate monooleate (Tween 80 or Montanox 80), may advantageously be used. Preferably, the PVA used has a molecular weight of from 30 to 200 kDaltons.

This non-ionic surfactant is, for example, hydrolysed to 88%. It is also particularly advantageous inasmuch as it increases the viscosity of the so-called “external” aqueous phase.

In the case of a double emulsion, the external aqueous phase advantageously also comprises at least one osmolarity agent in order to balance the osmotic pressure with the internal aqueous phase. The active ingredient is thus prevented from escaping towards the exterior medium.

An osmolarity agent normally used is glucose or any other sugar, such as mannitol and trehalose, but salts, such as sodium chloride, for example, may also be suitable.

The osmolarity agent is present in the external aqueous phase in principle in an amount sufficient to reach the ion concentration present in the internal aqueous phase. Generally, the concentration of osmolarity agent is then from 0.1 to 20% by weight relative to the weight of the aqueous phase. This salt is preferably used in the internal aqueous phase at a concentration of 0.6% (m/m) which is the concentration most suited to injectable preparations. Preferably, glucose is used in the external aqueous phase at a concentration of 11.5% (m/m) which is the amount necessary to equal the ion concentration present in the internal aqueous phase.

Finally, the aqueous phase of the emulsion advantageously contains at least one viscosity agent enabling the viscosity of the phase to be adjusted so that it is acceptable for the implementation of the second step described hereinafter. These agents also help to stabilize the double emulsions by limiting the coalescence of the drops in suspension.

The aqueous phase generally contains from 10 to 80%, preferably from 30 to 70%, preferentially from 40 to 60% by weight of viscosity agents relative to the total weight of the emulsion.

In general, the viscosity agent may be selected from hydrophilic polymers, such as glycol ethers and esters, poloxamers, such as Lutrol®, poly(aminosaccharides), such as chitins or chitosans, poly(saccharides), such as dextran, and the derivatives of cellulose, such as the Carbopols®.

Preferably, the viscosity agent is a poloxamer: block polymer of polyethylene/polypropylene. The hydrophobic central nucleus is constituted by polypropylene and is surrounded by hydrophilic sequences of polyethylene. Preferably, poloxamer 188 (Lutrol® F68, BASF), which forms gels at concentrations of from 50 to 60% in water, is used.

The amount of agent to be used depends on the viscosity to be reached. Preferably, however, the concentration of poloxamer is less than 50% by mass in order to prevent the formation of a gel.

The combination of stabilizing agents and viscosity agents has a very particular importance insofar as it has been demonstrated that the success of the laminar shearing step depends, in a large part, on the ratio of the viscosities between the dispersed phase and the continuous phase. The combination of these agents therefore makes it easy both to obtain the optimum viscosity ratio between the phases and to obtain a stability of the emulsion with respect to the coalescence of the drops.

The aqueous phase of the emulsion may comprise any other agent or additive normally present in pharmaceutical formulations, such as preservatives and buffer agents.

Especially, the internal aqueous phase may also comprise at least one other active ingredient, in particular a water-soluble active ingredient.

Thus, a hydrophilic active ingredient and a lipophilic active ingredient can be combined by dissolving the first in the internal aqueous phase and the second in the polymeric organic phase.

Finally, the aqueous phase(s) of the emulsion is(are) preferably saturated with organic solvent in order to prevent diffusion thereof from the organic phase towards those phases.

As described above, the microspheres can be prepared starting from a double emulsion in which a second aqueous phase (called “internal”) is dispersed in the polymeric organic phase.

The internal aqueous phase of the double emulsion may contain the agents already mentioned above in connection with the external aqueous phase.

The internal aqueous phase of the double emulsions may, however, also contain at least one protein, as surfactant, and/or viscosity agent and/or as active ingredient.

Thus, it may contain a high-molecular-weight protein, such as HSA (Human Serum Albumin), in order to increase the viscosity and/or to stabilize the emulsion. For it has been observed that the amphiphilic character of such macromolecules can help to stabilize the emulsion. Preferably, the internal aqueous phase comprises HSA or at least one protein at concentrations of from 0.01% to 10% by weight relative to the weight of the internal aqueous phase, preferably from 0.1 to 2%.

When the internal aqueous phase comprises a protein, it is then generally preferable to add other additives in order to form a medium suitable for the protein of interest, in particular as regards the pH. The presence of a buffer agent having a pH close to the pI of the protein advantageously enables the natural conformation of the protein to be preserved.

The internal aqueous phase of the double emulsions may therefore also contain the compounds necessary to form a buffer stabilizing the pH of the solution. The pH values suitable for the various proteins and the corresponding buffers are known by the person skilled in the art and will therefore not be specified here.

The internal aqueous phase may also contain stabilizing agents, such as poloxamer 188 described by SANCHEZ, A. et al. (Biodegradable micro- and nanoparticles as long-term delivery vehicles for interferon alpha. Eur. J. Pharm. Sci. (2003) 18, 221-229).

The internal aqueous phase advantageously also contains a cosurfactant. The latter, combined with the protein, is concentrated at the interface between the internal aqueous phase and the organic phase and helps to reduce the surface tension between those two media.

The cosurfactant used is preferably Solutol HS 15 from BASF. This product is a mixture of mono- and di-polyethylene glycol 660 esters of 12-hydroxystearic acid. It is soluble in water, ethanol and 2-propanol.

The internal aqueous phase comprises a surfactant at a concentration of from 0.01 to 10%, preferably from 0.05 to 1%, and more specifically from 0.1 to 0.2% by weight relative to the weight of the internal aqueous phase.

The internal aqueous phase of the microspheres may also advantageously contain an active ingredient.

This is found to be particularly valuable for the administration of fragile hydrophilic active ingredients, such as, for example, proteins or polypeptides, because it is often observed that there is a deterioration in the biological activity of those compounds when there is a change of chemical environment, such as, for example, dispersion in an organic solvent or if the temperature or pH varies.

In the preparation of the microspheres, the active ingredient will not undergo any deterioration in its activity because it will be dissolved in the internal aqueous phase of a double emulsion at the optimum pH. The changes in the physico-chemical environment and therefore the structural alterations of the molecule are thus reduced, which enables the activity of the active ingredient to be preserved.

The method for preparing the microspheres then comprises a second step which consists in subjecting the emulsion obtained to laminar shearing. The laminar shearing is preferably carried out in a Couette device. It is the viscoelasticity of the emulsion obtained owing to an optimum viscosity ratio between the phases present, the rate of rotation of the rotor and the rate of injection of the emulsion into the air gap which will define the size and uniformity of size of the microspheres obtained.

The method for preparing the microspheres then comprises a third step which consists in extracting the organic solvent from the dispersed polymer solution.

This step can be carried out by any method known to the person skilled in the art, for example evaporation under the effect of heat or under vacuum.

According to a preferred embodiment, it is carried out by extracting the organic solvent in water. More specifically, a large amount of water in which the organic solvent will diffuse is added to the prepared monodisperse emulsion. This embodiment has, in particular, the advantage of protecting the encapsulated active ingredient from variations in temperature or pressure.

As the solvent disappears from the organic phase by diffusion in the water, the polymer precipitates and, depending on the type of starting emulsion, forms microspheres having a polymer matrix retaining droplets of aqueous solution (double emulsion), or solid microspheres (single emulsion).

The precipitation preferably takes place with slight agitation in order to preserve the homogeneity of the emulsion and the suspension.

Finally, in a last step, the microspheres can be collected by the usual methods, for example by filtering the solution.

If necessary, the microspheres can then be lyophilized in the presence of a cryoprotector. Of the cryoprotective agents, polyols and electrolytes may especially be mentioned. In particular, for example, glycerin, mannose, glucose, fructose, xylose, trehalose, mannitol, sorbitol, xylidine and other polyols, and polyethylene glycol are suitable. Sodium chloride may be mentioned as an electrolyte.

Thus, the microspheres prepared can act as vehicles for one or more active ingredients, especially hydrophilic and lipophilic active ingredients, permitting their homogeneous and predetermined release over time.

The invention will be described in more detail in the Examples and Figures which follow and which show:

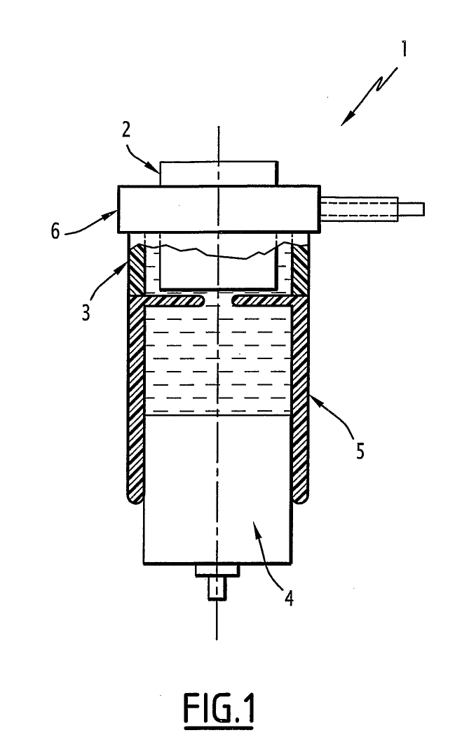

FIG. 1: a diagrammatic view of a Couette device;

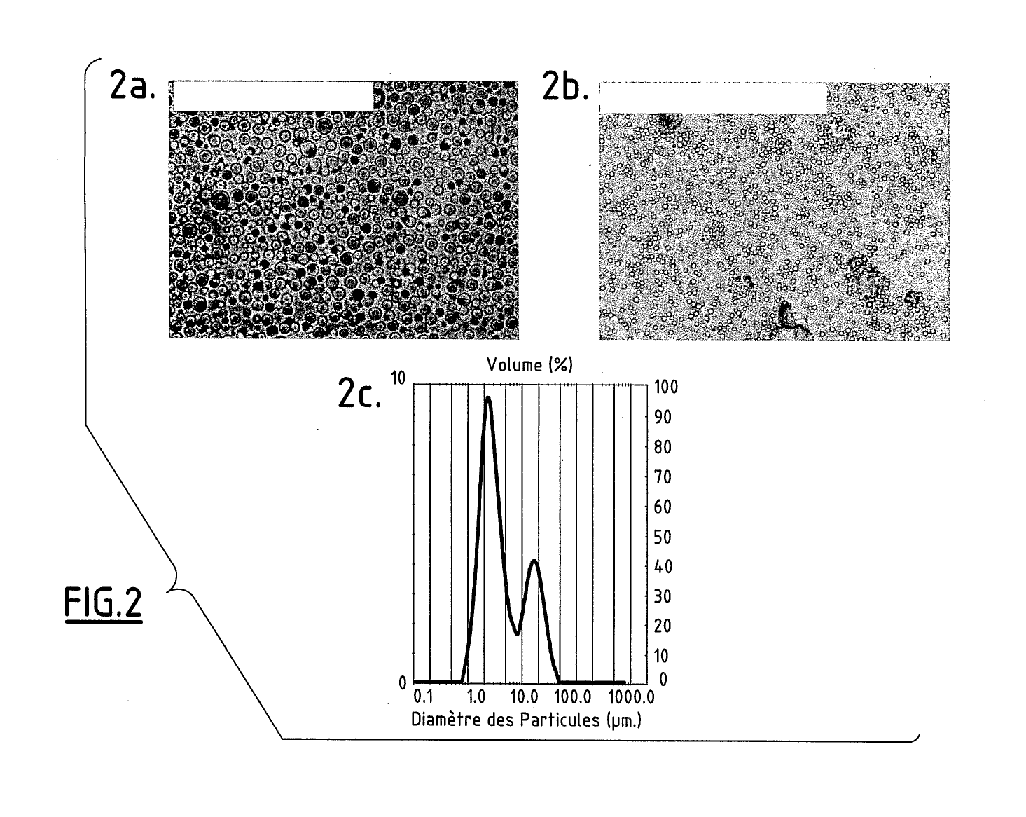

FIG. 2: optical microscopy photographs of microspheres prepared in accordance with Example 2 (a) before extraction (objective ×100); (b) after drying and redispersion (objective ×40); (c) their particle size distribution after redispersion;

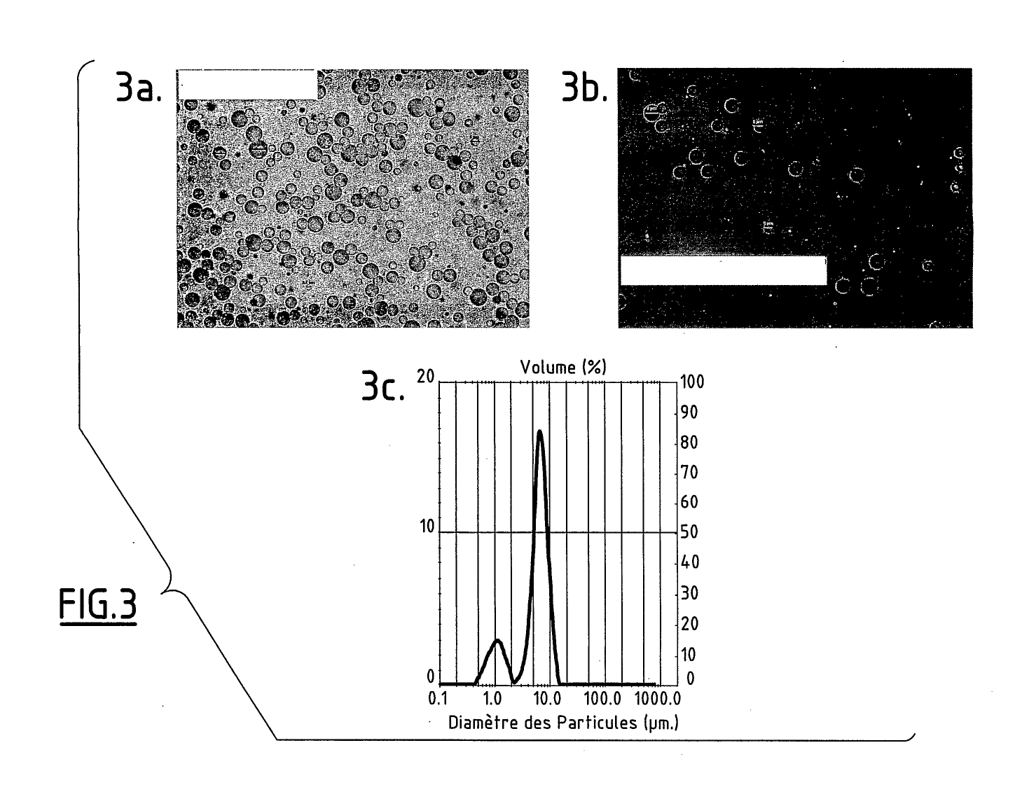

FIG. 3: optical microscopy photographs of polymer microspheres in accordance with Example 3 (a) before extraction (objective ×40); (b) after extraction (objective ×40); (c) their particle size distribution after redispersion;

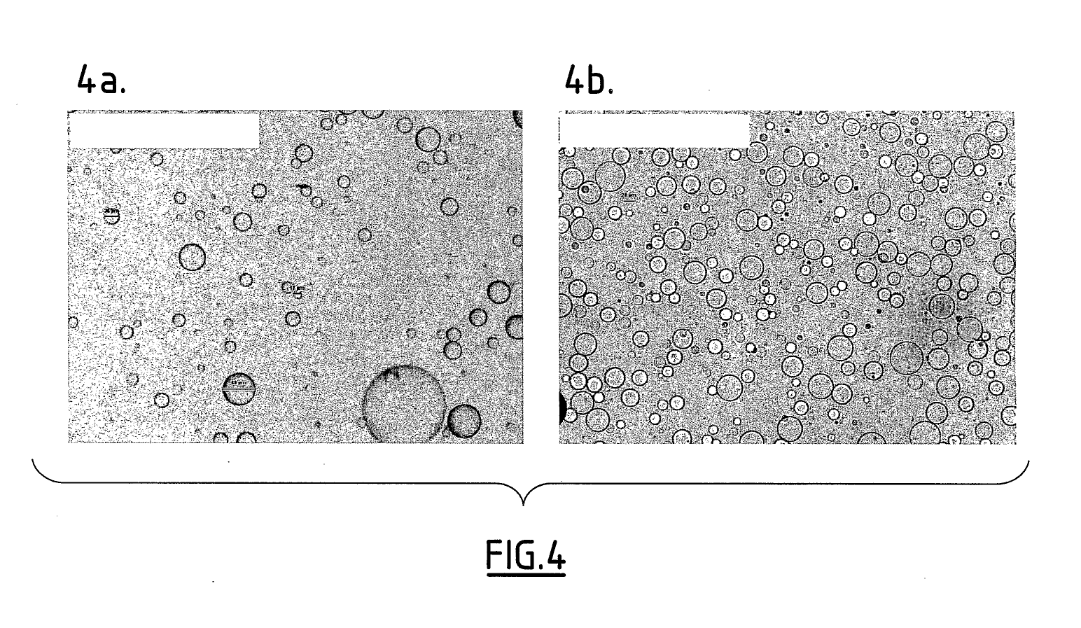

FIG. 4: optical microscopy photographs of an inverse emulsion in accordance with Example 5 (a) before shearing (objective ×10); (b) after shearing at 600 rpm using the Couette apparatus (objective ×10);

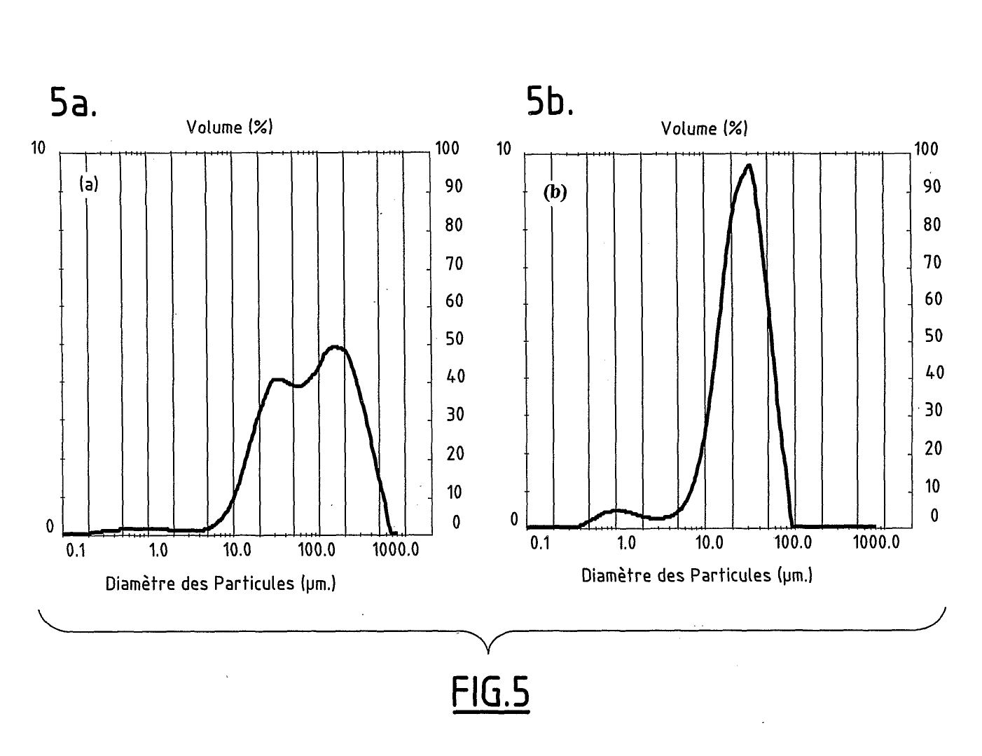

FIG. 5: particle size distribution of microspheres obtained (a) in accordance with Example 7, by standard random shearing (paddle agitator); (b) in accordance with Example 6, by laminar shearing (Couette apparatus).

### EXAMPLES

### Example 1

### General Procedure for the Preparation of Microspheres from a Single Emulsion

This method can be used to prepare biodegradable polymer microspheres which are useful, in particular, for the delivery of lipophilic active ingredients.

The active ingredient to be encapsulated is dispersed or dissolved in an organic phase composed of a PLGA dissolved in ethyl acetate.

This organic phase is then emulsified in an aqueous phase containing water and a hydrophilic surfactant, such as PVA, at from 0.1 to 10%, preferably from 1 to 4%, and a viscosity agent, such as a polyethylene glycol or a poloxamer, at from 10 to 50%.

The ratio of the viscosities of the two phases is adjusted in order to optimize the shearing efficiency. Preferably, the ratio between the viscosity of the organic phase and that of the aqueous phase is from 0.1 to 10, more precisely from 3 to 8.

The so-called “coarse” emulsion so obtained is then subjected to laminar shearing. This step is preferably carried out in a Couette device, shown in FIG. 1. The controlled shearing enables the drops of dispersed phase to be rendered monodisperse; however, it also enables their size to be controlled.

Preferably, the controlled shearing is carried out by placing the emulsion in contact with a moving solid surface, the speed gradient characterizing the flow of the emulsion being constant in a direction perpendicular to the moving solid surface. Such shearing may be effected, for example, in a cell constituted by two concentric cylinders rotating relative to each other, such as a “Couette” cell.

A Couette device (1) is shown in FIG. 1. It comprises a rotor (2), a stator (3) and a piston (4). The emulsion is introduced into the space defined between the rotor and the stator, called the air gap, by means of an injection syringe (5). The emulsion sheared between the rotor and the stator is then collected on passing out into a recovery vessel (6) in a sealed flask. The shearing rate, the width of the air gap and the injection rate are adjustable parameters which can be varied in accordance with the desired size of the microspheres.

For details of this method, see in particular applications WO 97/38787, FR 2767064 and WO0185319.

Once the emulsion has been rendered thus monodisperse, it is possible to proceed with the extraction of the solvent in order to precipitate the microspheres. The extraction is effected by adding a volume of water calculated in accordance with the solubility of the ethyl acetate in water and the amount of emulsion obtained. A volume of water equal to at least twice the minimum volume necessary to dissolve the ethyl acetate is preferably used.

Inasmuch as ethyl acetate is more soluble in water at low temperature, a second step of cold extraction is carried out in order to remove the solvent residues. Thus, after 30 minutes' agitation, a second volume of demineralized water cooled to 5° C. is added and the whole is maintained under agitation for another 30 minutes. The extraction of the solvent thus carried out is almost total.

At the end of the 30 minutes, the microspheres are then separated from the extraction medium by filtration under pressure on a nylon filter having a porosity of 0.45 μm. The cake recovered is rinsed 3 times with 1 litre of demineralized water. The microspheres are then left to dry overnight at ambient temperature or are frozen and lyophilized after the addition of a cryoprotective agent.

Once dry, the microspheres are redispersed in a solution of surfactant Montanox® 20 or 80 (BASF) at 1% (Montanox® 80: polysorbate monooleate and Montanox® 20: polysorbate monolaurate) by agitation and passage through an ultrasound bath. The redispersed microspheres are characterized by observation under a microscope and their size distribution is measured by laser granulometry.

### Example 2

### Preparation of 2.5 μm Microspheres from a Single Emulsion

In a flask, the continuous aqueous phase is prepared by dissolving at 70° C. 0.9 g of PVA in 14.14 g of demineralized water saturated with ethyl acetate (3%) under magnetic agitation. After cooling, 15 g of PEG 400 are incorporated therein. This aqueous phase therefore contains 3% of PVA, 50% of PEG 400 and is saturated with ethyl acetate.

The organic phase is prepared in a sealed flask by dissolving under magnetic agitation 2.6 g of PLGA 75/25 in 17.39 g of ethyl acetate saturated with water (3%). This organic phase therefore contains 13% of PLGA dissolved in ethyl acetate saturated with water.

All of this organic phase is then emulsified in 20 g of the above aqueous phase by manual agitation using a spatula. The emulsion contains 50% by mass of dispersed organic phase.

The premix so obtained is then placed in the Couette apparatus and sheared at a rate of 400 rpm in an air gap of 100 μm with an upstroke speed of the piston of 0.7 which corresponds to a flow rate of approximately 7 ml/min. The diameter of the rotor is 2 cm. FIG. 2(a) shows the homogeneous size distribution of the emulsion so prepared.

Once the emulsion has been rendered thus monodisperse, the solvent is extracted and the microspheres are filtered and then dried, as explained in Example 1. FIG. 2(b) shows the regular visual appearance of the microspheres obtained, after redispersion as described in Example 1.

The size distribution of the microspheres is measured by laser granulometry (see FIG. 2(c)); it is centred on 2.5 μm.

### Example 3

### Preparation of 6.5 μm Microspheres from a Single Emulsion

In a flask, the continuous aqueous phase is prepared by dissolving at 70° C. 1.2 g of PVA in 35.25 g of demineralized water saturated with ethyl acetate (3%) under magnetic agitation. After cooling, 4.02 g of PEG 2000 are incorporated therein. This aqueous phase therefore contains 3% of PVA and 10% of PEG 2000 and is saturated with ethyl acetate.

The organic phase is prepared in a sealed flask by dissolving under magnetic agitation 2.67 g of PLGA 75/25 in 17.89 g of ethyl acetate saturated with water (3%). This organic phase therefore contains 13% of PLGA dissolved in ethyl acetate saturated with water.

All of this organic phase is then emulsified in 20 g of the above aqueous phase by manual agitation using a spatula. The emulsion contains 50% by mass of dispersed organic phase.

The premix so obtained is then placed in the Couette apparatus and sheared at a rate of 300 rpm in an air gap of 100 μm with an upstroke speed of the piston of 0.7 which corresponds to a flow rate of approximately 7 ml/min. The diameter of the rotor is 2 cm. FIG. 3(a) shows the homogeneous size distribution of the emulsion so prepared.

Once the emulsion has been rendered thus monodisperse, the solvent is extracted and the microspheres are filtered and then dried, as explained in Example 1. FIG. 3(b) shows the regular appearance of the microspheres after extraction of the solvent as described in Example 1.

The size distribution of the microspheres is measured by laser granulometry (see FIG. 3(c)); it is centred on 6.5 μm.

### Example 4

### General Procedure for the Preparation of Microspheres from a Double Emulsion

This method is used for the preparation of polymer microspheres which are useful, in particular, for the delivery of hydrophilic active ingredients or a combination of a hydrophilic active ingredient and a lipophilic active ingredient.

First of all an inverse emulsion (W/O) is prepared by dispersing a so-called “internal” aqueous phase in an organic phase comprising a solution of polymer (PLGA 75/25, for example).

The ratio of the viscosities of the two phases, for the inverse emulsion, is adjusted in order to optimize the shearing efficiency. Preferably, the ratio between the viscosity of the internal aqueous phase and that of the organic phase is from 0.1 to 10, more precisely from 0.1 to 0.3.

The internal aqueous phase contains a protein, especially HSA, at from 0.01 to 10%, preferably from 0.1 to 2%, a co-surfactant, especially Solutol® HS15, at from 0.01 to 10%, preferably from 0.05 to 1% and a salt, especially sodium chloride, at from 0.1 to 20%, preferably 0.6%.

The organic phase is prepared in a sealed flask by dissolving, under magnetic agitation, PLGA 75/25 at from 5 to 30%, preferably 20%, in a solution of ethyl acetate saturated with water (3%).

Generally, the hydrophilic active ingredient to be encapsulated is contained in the internal aqueous phase and the lipophilic active ingredient in the organic phase.

The “coarse” inverse emulsion is then subjected to shearing as described in Example 1 in order to obtain a dispersed phase of controlled size and distribution. The controlled shearing step can be carried out using a Couette device or in a turbulent device of the Ultra-Turrax type.

In a flask, an aqueous solution of PVA at from 0.01 to 10%, preferably from 1 to 4%, is brought to 70° C. under magnetic agitation. After cooling, Lutrol® F68 at from 0.1 to 40%, preferably from 1 to 10%, and NaCl at a concentration identical to that of the internal aqueous phase: 0.6%, are added to the solution (external aqueous phase).

Subsequently, this so-called “external” aqueous phase is saturated with organic solvent, preferably ethyl acetate, which represents, for this particular solvent, a concentration of approximately 3% by weight relative to the weight of the aqueous phase.

The inverse emulsion is then incorporated in the external aqueous phase described above. This step can be carried out manually using a spatula.

The ratio of the viscosities of the two phases, in the case of the double emulsion, is adjusted in order to optimize the shearing efficiency. Preferably, the ratio between the viscosity of the organic phase and that of the external aqueous phase is from 0.1 to 10, more precisely from 3 to 8.

The emulsion so obtained is also called a “premix” or a “coarse” emulsion inasmuch as the dispersed phase is constituted by droplets of large and very variable size.

The “coarse” emulsion is then subjected to shearing as described in Example 1 in order to obtain a dispersed phase of controlled size and distribution. The controlled shearing step can be carried out using a Couette device.

Once the emulsion is rendered thus monodisperse, the solvent is extracted in order to precipitate the microspheres. The extraction is effected by adding a volume of water calculated in accordance with the solubility of the ethyl acetate in water and the amount of emulsion obtained. A volume of water equal to at least twice the minimum volume necessary to dissolve the ethyl acetate is preferably used.

Inasmuch as ethyl acetate is more soluble in water at low temperature, a second step of cold extraction is carried out in order to remove the solvent residues. Thus, after 30 minutes' agitation, a second volume of demineralized water cooled to 5° C. is added and the whole is maintained under agitation for another 30 minutes. The extraction of the solvent thus carried out is almost total.

The monodisperse microspheres containing the active ingredient(s) are filtered and lyophilized as described in Example 1.

Once dry, the microspheres are redispersed in a solution of surfactant Montanox® 20 or 80 (BASF) at 1% (Montanox® 80: polysorbate monooleate and Montanox® 20: polysorbate monolaurate) by agitation and passage through an ultrasound bath. The redispersed microspheres are characterized by observation under a microscope and their size distribution is measured by laser granulometry.

### Example 5

### Preparation of an Inverse Emulsion of 1 μm

In a flask, the internal aqueous phase is prepared under magnetic agitation. It is composed of 0.04 g of HSA, 0.0036 g of Solutol® HS15 and 0.022 g of NaCl dissolved in 4 g of citrate buffer pH5 saturated with ethyl acetate (3%). This internal aqueous phase therefore contains 1% of HSA, 0.1% of Solutol® HS15 and is saturated with ethyl acetate.

The organic phase is prepared in a sealed flask by dissolving, under magnetic agitation, 3.2 g of PLGA 75/25 in 12.82 g of ethyl acetate saturated with water (3%). This organic phase therefore contains 20% of PLGA dissolved in ethyl acetate saturated with water.

The internal aqueous phase is dispersed manually in the ethyl acetate solution using a spatula in order to obtain a coarse inverse emulsion.

This emulsion contains 20% by weight of internal aqueous phase relative to its total weight. The stability of the coarse emulsion produced is verified before shearing by the absence of phase separation and coalescence.

The premix so obtained is then placed in the Couette apparatus and sheared at a rate of 400 rpm in an air gap of 100 μm with an upstroke speed of the piston of 0.7 which corresponds to a flow rate of approximately 7 ml/min. The diameter of the rotor is 2 cm. The inverse emulsion is stable after shearing using the Couette apparatus.

The visual appearance, under a microscope, of the premix and the emulsion after shearing on the Couette apparatus is shown in FIGS. 4(a) and (b).

The calibrated double emulsion is then prepared as follows.

### Example 6

### Preparation of 28 μm Monodisperse Microspheres from a Double Emulsion

First of all, an inverse emulsion is prepared as in Example 5 with:
20% of PLGA in the ethyl acetate;
1% of HSA in the internal aqueous phase;
0.1% of Solutol® HS15;
0.6% of NaCl in the internal aqueous phase.

The coarse inverse emulsion obtained is then sheared using the Ultra-Turrax (power 24000) for 3 minutes or else in the Couette device at 400 rpm.

20 g of inverse emulsion obtained are then incorporated using a spatula in the same amount of external aqueous phase composed of 3 g of Lutrol® F68, 0.9 g of PVA and 0.18% of NaCl. This external aqueous phase therefore contains 10% of Lutrol® F68, 3% of PVA and 0.6% of NaCl and is saturated with ethyl acetate. This double emulsion contains 50% by weight of inverse emulsion relative to its total weight.

The premix so obtained is then placed in the Couette apparatus and sheared at a rate of 100 rpm in an air gap of 100 μm with an upstroke speed of the piston of 0.7 which corresponds to a flow rate of approximately 7 ml/min. The diameter of the rotor is 2 cm.

The double emulsion collected at the outlet of the apparatus is diluted under agitation in 250 ml of saline (0.6% NaCl) at ambient temperature.

After 10 minutes, a second volume of 250 ml of saline is added at 5° C. and agitation is continued for 10 minutes. The conversion of the double globules into solid microspheres is observed. The microspheres are then separated from the extraction medium by filtration under pressure on a nylon filter having a porosity of 0.45 μm. The cake recovered is rinsed 3 times with 1 litre of demineralized water.

For lyophilization, the filtered microspheres are dispersed in a trehalose solution. The percentage of trehalose added corresponds to 5% of the microspheres to be lyophilized. The sample is first of all frozen in liquid nitrogen, then stored in a freezer at −24° C. The lyophilization is carried out in accordance with the following ramp with a vacuum fixed at 0.12 mbar:
‰ primary desiccation by passing from −44° C. to −10° C. in 4 hours and isothermal desiccation at −10° C. for 15 hours 30 minutes.
‰ secondary desiccation by passing from −10° C. to +10° C. in 30 minutes and return to ambient temperature in 30 minutes.

Once dry, the microspheres are redispersed in a solution of surfactant Montanox® 20 or 80 (BASF) at 1% (Montanox® 80: polysorbate monooleate and Montanox® 20: polysorbate monolaurate) by agitation and passage through an ultrasound bath. The redispersed microspheres are characterized by observation under a microscope and their size distribution is measured by laser granulometry. The size distribution of the microspheres is centred on 28 μm (FIG. 5b).

### Example 7

### Preparation of Microspheres by Turbulent Shearing

A batch of microspheres was prepared in accordance with Example 6, using shearing in turbulent operation (Ultra-Turrax then paddle agitation) instead of the laminar shearing brought about by the Couette apparatus.

The size distribution of these microspheres was evaluated by a laser granulometer (FIG. 5a) and compared with that established for the microspheres prepared in accordance with Example 6 (FIG. 5b).

It is readily observed that the laminar shearing such as provided by the Couette device enables a narrower size distribution and therefore a more pronounced monodisperse character to be obtained. As a result, the release kinetics of the active ingredients contained in the microspheres is better controlled.

1-20. (canceled)

21. A method for preparing monodisperse biodegradable microspheres comprising the steps of:
a) preparing an emulsion comprising at least one polymer phase, which comprises an active ingredient, and at least one aqueous phase, the viscosity of the organic phase and the aqueous phase having a ratio of from 0.1 to 10;
b) subjecting the emulsion obtained to controlled laminar shearing;
c) removing the solvent from the polymer phase; and
d) isolating the microspheres so obtained.

22. The method of claim 21, wherein the majority of the microspheres are constituted in majority by a biodegradable polymer.

23. The method of claim 22, wherein the biodegradable polymer is selected from poly(α-hydroxy) acids, the aliphatic polyesters of poly(α-hydroxy acids), of poly(ε-caprolactones)—PCL, of polydioxanones—PDO, polyorthoesters, polyanhydrides, polycyanoacrylates, polyurethanes, polypeptides or poly(amino acids), modified polysaccharides, cellulose, polycarbonates, polydimethylsiloxanes and poly(vinyl acetates) and their derivatives and copolymers.

24. The method of claim 22, wherein the biodegradable polymer is selected from polylactic acids (PLA), and the copolymers of polylactic acid/polyglycolic acid (PLGA).

25. The method of claim 21, wherein the polymer has a molecular weight of from 50 to 500 kDaltons.

26. The method of claim 21, wherein the organic solvent of the organic phase of the emulsion is ethyl acetate.

27. The method of claim 21, wherein the active ingredient is lipid-soluble.

28. The method of claim 21, wherein the active ingredient is water-soluble.

29. The method of claim 21, wherein the active ingredient is a peptide or a protein.

30. The method of claim 21, wherein the emulsion prepared in step (a) comprises a hydrophilic active ingredient in combination with a lipophilic active ingredient.

31. The method of claim 21, wherein the organic phase of the emulsion represents from 10 to 60% by weight relative to the total weight of the emulsion.

32. The method of claim 21, wherein the organic phase of the emulsion comprises from 1 to 50%, preferably from 5 to 30% by weight of polymer.

33. The method of claim 21, wherein the organic phase of the emulsion comprises from 1 to 50%, preferably from 5 to 30% by weight of active ingredient.

34. The method of claim 21, wherein the emulsion is a double emulsion.

35. The method of claim 21, wherein the external and/or internal aqueous phase of the emulsion contains at least one stabilizing agent and/or at least one viscosity agent.

36. The method of claim 21, wherein the external and/or internal aqueous phase of the emulsion contains at least one stabilizing agent and/or at least one osmolarity agent and/or at least one surfactant and/or at least one buffer agent.

37. The method of claim 21, wherein the step of calibration by laminar shearing is carried out in a Couette device.

38. The method of claim 21, wherein the step of removing the solvent from the polymer phase is carried out by extraction in water.

39. A method for the administration of active ingredients in the human or animal organism, making use of the microspheres that can be obtained according to claim 21.

40. The method of claim 39, wherein the active ingredient is selected from antibiotics, hypolipidaemics, antihypertensives, antiviral agents, beta blockers, bronchodilators, cytostatics, psychotropic agents, hormones, vasodilators, anti-allergics, analgesics, antipyretics, antispasmodics, anti-inflammatories, anti-angiogenics, antibacterials, anti-ulcerants, antifungals, anti-parasitics, antidiabetics, anti-epileptics, anti-Parkinsons, antimigraines, anti-Alzheimers, anti-acneics, antiglaucomic agents, anti-asthmatics, neuroleptics, antidepressants, anxiolytics, hypnotics, normothymics, sedatives, psychostimulants, anti-osteoporosis agents, anti-arthritics, anticoagulants, antipsoriasis agents, hyperglycaemics, orexigenics, anorexigenics, anti-asthenics, anticonstipation agents, antidiarrhoeals, anti-trauma agents, diuretics, myorelaxants, enuresis medicaments, erection disorder medicaments, vitamins, peptides, proteins, anticancer agents, nucleic acids, RNA, oligonucleotides, ribozymes and DNA.
